(19) United States
(12) Patent Application Publication
(10) Pub. No.: US 2019/0161719 A1
(43) Pub. Date: May 30, 2019

(54) LIQUID FEEDING DEVICE AND METHOD FOR FEEDING LIQUID

(71) Applicant: FURUKAWA ELECTRIC CO., LTD., Tokyo (JP)

(72) Inventors: Kenichi KIMURA, Tokyo (JP); Ken TSUKII, Tokyo (JP); Toru TAKAHASHI, Tokyo (JP); Mariko MATSUNAGA, Tokyo (JP); Jie XU, Tokyo (JP)

(73) Assignee: FURUKAWA ELECTRIC CO., LTD., Tokyo (JP)

(21) Appl. No.: 16/262,316

(22) Filed: Jan. 30, 2019

Related U.S. Application Data
(63) Continuation of application No. PCT/JP2018/015052, filed on Apr. 10, 2018.

(30) Foreign Application Priority Data
Apr. 10, 2017 (JP) 2017-077252

(51) Int. Cl. C12M 1/00; C12M 1/32

(52) U.S. Cl. CPC C12M 29/26; C12M 23/12

(57) ABSTRACT

Provided is a liquid feeding device which can improve collection efficiency of fine particles on an individual particle basis while improvement of workability is realized.

A liquid feeding device includes: an introduction portion disposed on one end portion of the liquid feeding device, and configured to introduce a liquid into a closed space; a discharge portion disposed on another end portion of the liquid feeding device, and configured to discharge the liquid supplied into the closed space; and an upper wall provided so as to oppose the upper surface of the chip, and configured to define the closed space. The upper wall has an inclined surface which is provided such that a gap formed between the inclined surface and the chip is reduced from an introduction portion side toward a discharge portion side.

### CROSS REFERENCE TO RELATED APPLICATIONS

This application based on International Patent Application No. PCT/JP2018/015052 filed Apr. 10, 2018, which claims the benefit of Japanese Patent Applications No. 2017-077252 filed Apr. 10, 2017, the full contents of both of which are hereby incorporated by reference in their entirety.

### BACKGROUND

### Technical Field

The present disclosure relates to a liquid feeding device which is placed on a chip, on which wells are formed, each storing a fine particle, such as a cell on an individual particle basis, thus being capable of forming a closed space between the liquid feeding device and the chip.

### Background

Conventionally, a fine particle screening device is widely used as a device for identifying and sorting minute object specimens, such as cells in carrying out research and inspections in the medical field. Recently, in research and inspection agencies, there is a demand for an increase in the efficiency of research and inspection by realizing identification and sorting without causing breakage of a specimen and, at the same time, by accurately performing these processes. Particularly, in a predetermined field, there is a high demand for identifying and sorting cells on an individual cell basis and hence, improvement in accuracy and improvement in efficiency are also required in performing these identification processes and sorting process on an individual cell basis.

For example, as one example of a method for accurately and efficiently identifying and sorting cells on an individual cell basis, a method is used where a plurality of cells are stored, on a one-to-one basis, in a plurality of minute wells, referred to as microchambers, which are formed on a chip. With such a method, measurement sensitivity of a screening device is increased and, at the same time, only target specimens can be collected using a capillary which can suck and deliver a cell on an individual cell basis. Further, an amount of wastage of target specimens contained in a cell suspension can be reduced.

As a conventional liquid feeding device which is applied to the above-mentioned microchamber chip, a cell expansion device is proposed which includes: a chip on which a plurality of microwells are formed; a flow-path-forming frame body disposed so as to form a micro flow path on a plurality of microchamber chips; an inlet portion formed in the flow-path-forming frame body; and an outlet portion formed in the flow-path-forming frame body so as to discharge, from the micro flow path, a cell suspension introduced into the micro flow path through the inlet portion (Japanese Patent No. 5825460). In this device, the micro flow path is formed on the chip, and a liquid flow substantially parallel to an upper surface of the chip is generated by the micro flow path.

Further, as another conventional liquid feeding device, a cell culture device is proposed which includes a body, and a lid portion detachably mounted on an upper portion of the body, wherein a culture chamber is formed in the body, and a medium discharge flow path is formed on the lid portion, and a porous filter is disposed between the body and the lid portion (Japanese Patent No. 5686310). According to this device, when cell culture is performed with the culture chamber coated with a scaffold material (carrier for cell culture), the scaffold material which is peeled off during usage can be captured by the porous filter.

### DISCLOSURE

When the flow velocity of a reagent is not increased in feeding the reagent or a cleaning solution onto a chip, variation may be caused in reaction, or the reagent may not react to cells in a uniform manner. When the flow velocity of the cleaning solution is low, a cleaning power is insufficient so that cells which are not stored in wells cannot be cleaned. For this reason, it is necessary to increase a flow velocity on a chip. However, in the technique of Patent Literature 1, when the flow velocity of a liquid is increased to a certain level or more, cells stored in wells may float up and escape, thus causing a problem where collection efficiency of cells which form target specimens is reduced.

Patent Literature 2 only discloses the technique having the structure where one hole portion formed in a body forms a culture chamber, and the culture chamber forms a closed space when the lid portion is closed. Accordingly, this technique is fundamentally different from a configuration of feeding a liquid onto a chip on which a plurality of wells are formed, which can store a plurality of cells on a one-to-one basis.

Particularly, when rare cells are collected as target specimens, for example, a small number of cells are to be collected from several hundreds of thousands of cells and hence, to make the target specimens objects to be collected, it is extremely important to increase the number of cells stored in the plurality of wells on the chip on an individual cell basis. To increase the number of cells stored, usually, for example, a cell suspension containing cells 1.5 to 2 times as many as the number of wells is introduced onto the chip so as to store the cells in the plurality of wells on an individual cell basis, and remaining cells which are not stored in the wells are removed from the chip by introducing a cleaning solution onto the upper surface of the chip. However, in the above-mentioned conventional technique, at the time of cleaning the chip, the cells stored in the respective wells may also escape and be removed from the wells by the cleaning solution, thus causing a reduction in the above-mentioned number of cells stored on the chip. Accordingly, there is a problem that rare cells cannot be efficiently collected so that valuable cells are lost.

Further, when the upper surface of the chip is cleaned with a cleaning solution in a state where cells are stored in wells in an open space, sucking the cleaning solution on the upper surface of the chip generates a liquid flow in the direction parallel to the upper surface of the chip in order to remove cells on the chip which are not stored in the wells. However, with this method, the cleaning solution flows through an area of easy flow due to open space and hence, in the vicinity of an upper surface of a chip having a large resistance, a sufficient liquid flow is only generated at the moment at which the liquid passes through the area, therefore the cells on the chip cannot be sufficiently removed by performing cleaning only once. Accordingly, it is necessary to repeatedly perform cleaning a plurality of times where a cleaning solution is supplied onto the upper surface of the chip again, and the cleaning solution on the upper surface of the chip is sucked. However, due to the open space, an upwardly swirling liquid flow is easily generated in the wells in supplying a cleaning solution onto the upper surface of the chip and, as a result, the cells stored in the wells may escape. That is, cleaning is repeatedly performed due to insufficient cleaning power. However, as the number of cleaning times is increased, the number of cells stored in the wells is reduced, thus causing a reduction and loss in collection efficiency of rare cells.

The present disclosure is related to providing a liquid feeding device and a method for feeding a liquid which can improve collection efficiency of fine particles on an individual particle basis while improvement of workability is realized.

### SUMMARY

The present disclosure is directed to a liquid feeding device capable of forming a closed space between the liquid feeding device and an upper surface of a chip on which at least one well is formed for storing a fine particle, the liquid feeding device comprising: an introduction portion disposed on one end side of the liquid feeding device, and configured to introduce a liquid into the closed space; a discharge portion disposed on another end side of the liquid feeding device, and configured to discharge the liquid supplied into the closed space; and an upper wall provided so as to oppose the upper surface of the chip, and configured to define the closed space, wherein the upper wall has an inclined surface which is provided such that a gap formed between the inclined surface and the chip is reduced from an introduction portion side toward a discharge portion side.

A closed flow path of a liquid which flows from the introduction portion side toward the discharge portion side is formed in the closed space.

The closed flow path generates a liquid flow which flows from the inclined surface toward the upper surface of the chip.

It is preferable that the inclined surface be a tilted surface having a gradient with respect to the upper surface of the chip, and a cross-sectional area of the closed space in a direction perpendicular to a tilt direction of the tilted surface be gradually reduced from the introduction portion side toward the discharge portion side.

It is preferable that the chip have a plurality of wells arranged in an aligned manner on the upper surface of the chip, and the inclined surface be formed at a position which corresponds to all of the plurality of wells as viewed in a plan view.

The introduction portion has: a supply port into which a liquid is supplied from an outside; a chamber which is coupled to the supply port, and which extends in a width direction of the closed space as viewed in a plan view; and a first slit portion which makes the chamber and the closed space communicate with each other, and which extends along the width direction of the closed space as viewed in a plan view.

It is preferable that a height of the first slit portion be set smaller than a maximum height of the closed space.

The discharge portion has: a discharge port which is coupled to the closed space, and through which a liquid in the closed space is discharged to an outside; and a second slit portion which is formed between the closed space and the discharge port, and which extends upward from the closed space.

The liquid feeding device further includes a frame-shaped leg portion which abuts on the upper surface of the chip, and which is provided so as to surround the closed space, and the frame-shaped leg portion includes: a pair of side leg portions disposed on both sides of the closed space along a direction from the introduction portion side toward the discharge portion side; and an introduction-side leg portion disposed on the introduction portion side, and an intermittent portion is formed in the discharge portion side of the frame-shaped leg portion.

The intermittent portion formed in the frame-shaped leg portion forms the discharge port, and the discharge port is provided between the pair of side leg portions on the discharge portion side of the pair of side leg portions.

The liquid feeding device further includes a pressing surface on a surface of the liquid feeding device which is not in contact with the chip, the pressing surface being capable of being pressed toward the upper surface of the chip, and the pressing surface is disposed on the discharge portion side.

It is preferable that the liquid feeding device be integrally molded by an elastic material.

The present disclosure is directed to a method for feeding a liquid where a closed space which allows introduction and discharge of a liquid is formed on an upper surface of a chip on which at least one well is formed for storing a fine particle, thus allowing introduction and discharge of the liquid onto and from the upper surface of the chip, and the method for feeding a liquid is including a closed space is formed such that the closed space is reduced from a side where the liquid is introduced toward a side where the liquid is discharged, and the liquid is introduced into the closed space, thus forming a closed flow path which flows from the introduction side toward the discharge side in the closed space.

In the method for feeding a liquid, a liquid flow which flows toward the upper surface of the chip is generated by the closed flow path.

### EFFECTS OF DISCLOSURE

According to the present disclosure, collection efficiency of fine particles on an individual particle basis can be improved while improvement of workability is realized.

### BRIEF DESCRIPTION OF DRAWINGS

FIG. 1A to FIG. 1C are perspective views schematically showing a configuration of a liquid feeding device according to an embodiment of the present disclosure, wherein FIG. 1A is an overall view as viewed from above, FIG. 1B is an overall view as viewed from below, and FIG. 1C is a view showing a cross section taken along line I-I in FIG. 1A.

FIG. 2A and FIG. 2B are views for describing a closed flow path formed in a closed space on a chip by the liquid feeding device shown in FIG. 1, wherein FIG. 2A is a cross-sectional view, and FIG. 2B is a plan view.

FIG. 3 is a partial cross-sectional view for describing a liquid flow generated in the vicinity of a well in the closed flow path shown in FIG. 2.

FIG. 4 is a perspective view showing a holder which fixes the liquid feeding device shown in FIG. 1 on the chip.

FIG. 5 is an exploded perspective view showing a fixing device which fixes the holder shown in FIG. 4 at the time of feeding a liquid onto the chip using the liquid feeding device.

FIG. 6A to FIG. 6E are cross-sectional views for describing a method for feeding a liquid using the liquid feeding device shown in FIG. 1.

FIG. 7 is a perspective view for describing a screening device which identifies and sorts target specimens from cells on the chip onto which a liquid is fed using the method for feeding a liquid shown in FIG. 6.

FIG. 8A to FIG. 8C are cross-sectional views for describing a variant of the method for feeding a liquid using the liquid feeding device shown in FIG. 1.

FIG. 9A to FIG. 9D are cross-sectional views for describing another variant of the method for feeding a liquid using the liquid feeding device shown in FIG. 1.

FIG. 10A and FIG. 10B are cross-sectional views for describing a variant of the liquid feeding device shown in FIG. 1.

### DETAILED DESCRIPTION

Hereinafter, an embodiment of the present disclosure will be described in detail with reference to drawings.

[Configuration of the Liquid Feeding Device]

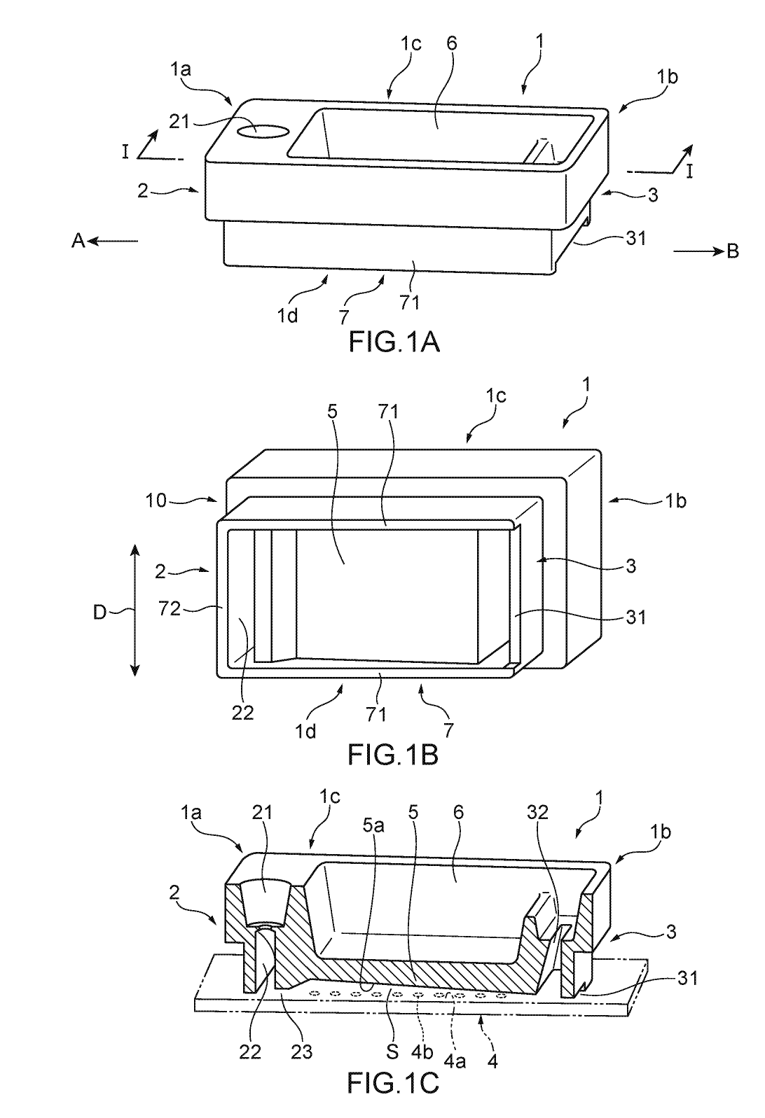

FIG. 1 is a perspective view schematically showing a configuration of a liquid feeding device according to the embodiment of the present disclosure, wherein FIG. 1A is an overall view as viewed from above, FIG. 1B is an overall view as viewed from below, and FIG. 1C is a view showing a cross section taken along line I-I in FIG. 1A. The liquid feeding device 1 shown in FIG. 1 is a device capable of forming a closed space between the liquid feeding device and an upper surface of a chip on which at least one well is formed. The configuration of the liquid feeding device shown in FIG. 1 merely shows one example, and the liquid feeding device of the present disclosure is not limited to the configuration shown in FIG. 1.

As shown in FIG. 1A to FIG. 1C, the liquid feeding device 1 includes: an introduction portion 2; a discharge portion 3; and an upper wall 5. The introduction portion 2 is disposed on one end portion 1a (the side indicated by an arrow A in the drawing) of the liquid feeding device, and is configured to introduce a liquid into a closed space S. The discharge portion 3 is disposed on another end portion 1b (the side indicated by an arrow B in the drawing) of the liquid feeding device 1, and is configured to discharge the liquid supplied into the closed space S. The upper wall 5 is provided so as to oppose an upper surface 4a of a chip 4, and is configured to define the closed space S. The liquid feeding device 1 is integrally molded by an elastic material, such as a resin or an elastomer. The above-mentioned elastic material may be a silicone resin (PDMS: dimethylpolysiloxane), for example.

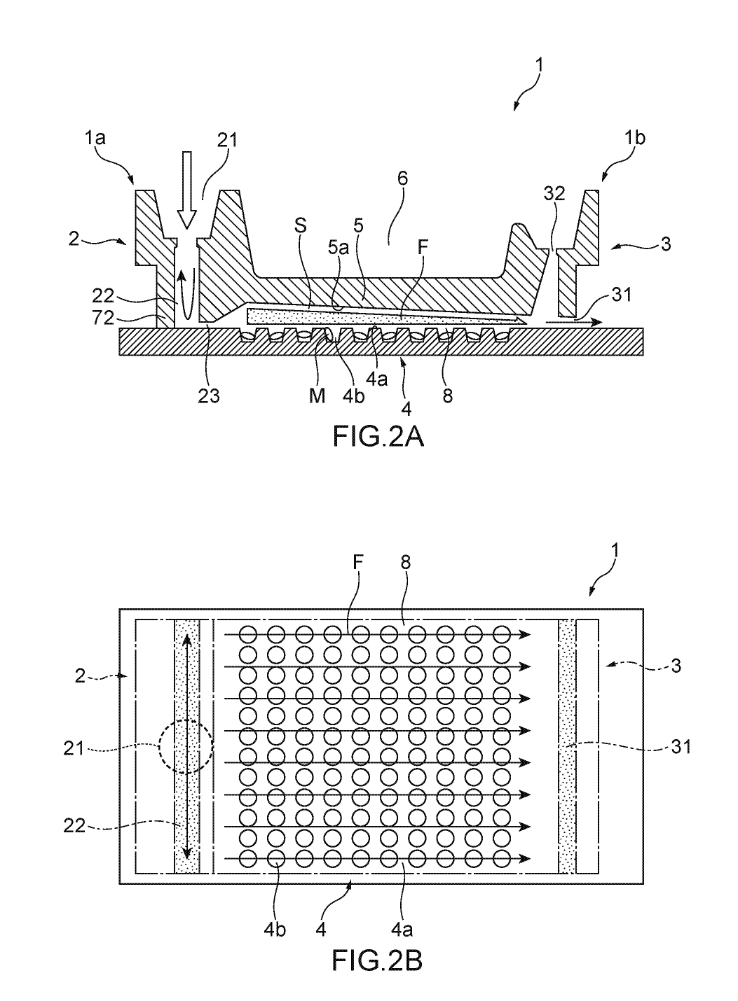

The chip 4 uses the liquid feeding device 1. The upper surface 4a of the chip 4 has a plurality of wells 4b, 4b, . . . arranged in an aligned manner, and the wells 4b, 4b, . . . can store fine particles, such as cells, on a one-to-one basis (see FIG. 2). In the present embodiment, the upper surface 4a means a surface disposed on the side to which fine particles are charged so as to be stored. The aligned arrangement of the plurality of wells may be in a matrix pattern or in a honeycomb pattern (honeycomb structure), for example. It is preferable that the chip 4 be a microwell chamber or an immunochamber, for example, and each well 4b has a size which corresponds to one cell. It is preferable to set the ratio of the well diameter to the cell diameter to between 1.0 and 3.0, and it is more preferable to set the ratio to between 1.5 and 2.0. In feeding a liquid onto the upper surface 4a of the chip 4, the liquid feeding device 1 is disposed so as to cover the upper surface 4a of the chip 4 including the plurality of wells 4b, 4b, . . . . Accordingly, the closed space S is formed between the upper surface 4a of the chip 4 and the upper wall 5 of the liquid feeding device 1.

The introduction portion 2 has a supply port 21, a chamber 22, and a first slit portion 23. A liquid is supplied into the supply port 21 from the outside. The chamber 22 is coupled to the supply port 21, and extends in the width direction D of the closed space S as viewed in a plan view, that is, when the liquid feeding device 1 is viewed from an upper portion 1c side in a state where the liquid feeding device 1 is placed on the chip 4 installed substantially horizontally. The first slit portion 23 makes the chamber 22 and the closed space S communicate with each other, and extends along the width direction D of the closed space S as viewed in a plan view.

The supply port 21 is formed of a circular hole formed in an upper portion 1c at one end portion 1a of the liquid feeding device 1. A device, such as a tubular member, is inserted into the supply port 21, and a liquid, such as a buffer (buffer solution), a cell suspension, a cleaning solution, or a reagent, is supplied to the supply port 21 from the outside.

The chamber 22 is formed of a rectangular parallelepiped recessed portion which is formed on a lower portion 1d at one end portion 1a of the liquid feeding device 1. An upper portion of the chamber 22 is coupled to the supply port 21, and a side portion of the chamber 22 on the other end portion 1b side is coupled to the first slit portion 23. In a state where the liquid feeding device 1 is placed on the chip 4, the chamber 22 can temporarily hold a liquid supplied from the supply port 21, and can gradually supply the liquid in the chamber 22 to the first slit portion 23.

The first slit portion 23 is formed of a recessed portion formed on the lower portion 1d at one end portion 1a of the liquid feeding device 1. A side portion of the first slit portion 23 on one end portion 1a side is coupled to the chamber 22, and a side portion of the first slit portion 23 on the other end portion 1b side is coupled to the closed space S. In a state where the liquid feeding device 1 is placed on the chip 4, it is preferable that the height of the first slit portion 23 from the upper surface 4a of the chip 4 be set smaller than the maximum height of the closed space S (see FIG. 2). With such a configuration, a liquid supplied from the chamber 22 is restricted when passing through the first slit portion 23 so that a liquid flow F with substantially the same velocity in the width direction D of the closed space S can be generated in the closed space S. In other words, a weir portion, having a slight clearance between the weir portion and the upper surface 4a of the chip 4, is formed on the introduction portion 2, and the slight clearance between the upper surface 4a of the chip 4 and the weir portion forms the first slit portion 23. With the formation of this weir portion, a liquid flow which flows from the chamber 22 to the first slit portion 23 is restricted. The size of the clearance is decided depending on the length of the clearance in the width direction D and a resistance of the flow path. For example, when the length of the clearance in the width direction D is set to approximately 15 mm, it is preferable to set the clearance to 1 mm or less, and it is more preferable to set the clearance to 0.5 mm or less.

The discharge portion 3 has a discharge port 31 and a second slit portion 32. The discharge port 31 is coupled to the closed space S, and discharges a liquid in the closed space S therethrough. The second slit portion 32 is formed between the closed space S and the discharge port 31, and extends upward from the closed space S.

The discharge port 31 is formed of a through hole formed in a frame-shaped leg portion described later. The discharge port 31 does not prevent a liquid flow in the closed space S, and the liquid in the closed space S is discharged to the outside through the discharge port 31.

The second slit portion 32 is formed of a through hole formed on the other end portion 1b side of the liquid feeding device 1. The lower portion of the second slit portion 32 is coupled to the closed space S, and the upper portion of the second slit portion 32 is coupled to a recessed portion 6 formed on the upper portion 1c of the liquid feeding device 1. Air accumulating in the closed space S is discharged to the outside through the discharge port 31 and the second slit portion 32 along with the liquid flow through the closed space S. It is preferable that the second slit portion 32 extend along the width direction D of the closed space S as viewed in a plan view. It is also preferable that the second slit portion 32 extend across the entire width of the closed space S. With such a configuration, air accumulating in the closed space S can be discharged with certainty.

The upper wall 5 has an inclined surface 5a which is provided such that a gap formed between the inclined surface 5a and the chip 4 is reduced from the introduction portion 2 side toward the discharge portion 3 side. It is preferable that the inclined surface 5a is a tilted surface having a gradient with respect to the upper surface 4a of the chip 4. It is also preferable that the cross-sectional area of the closed space S in the direction perpendicular to the tilt direction of the tilted surface is gradually reduced from the introduction portion 2 side toward the discharge portion 3 side.

The inclined surface 5a is formed of a uniform flat surface having a fixed inclination, or a plurality of flat surfaces having different inclinations, for example. In this case, it is preferable that the degree of the inclinations of the plurality of flat surfaces increase from the introduction portion 2 side toward the discharge portion 3 side. The inclined surface 5a is not limited to the flat surface. The inclined surface 5a may have a curved surface, or both of a curved surface and a flat surface, or may have a minutely uneven surface, such as a surface having a substantially corrugated shape in cross section.

It is preferable that the inclined surface 5a be formed at the position which corresponds to all of the plurality of wells 4b as viewed in a plan view, that is, when the liquid feeding device 1 is viewed from the upper portion 1c side in a state where the liquid feeding device is placed on the chip 4 installed substantially horizontally. To be more specific, it is preferable that the inclined surface 5a be formed over the entire upper wall 5 in the direction from the introduction portion 2 side toward the discharge portion 3 side, and be also formed over the entire upper wall 5 in the direction (width direction) perpendicular to the direction from the introduction portion 2 side toward the discharge portion 3 side. Provided that the inclined surface 5a is disposed at the position which corresponds to the plurality of wells 4b on the chip 4, that is, the inclined surface 5a is disposed just above the plurality of wells 4b, the inclined surface 5a may be formed on the part of the upper wall 5.

The liquid feeding device 1 also includes a frame-shaped leg portion 7 which abuts on the upper surface 4a of the chip 4, and is provided so as to surround the closed space S. The frame-shaped leg portion 7 includes a pair of side leg portions 71, 71 disposed on both sides of the closed space S along the direction from the introduction portion 2 side toward the discharge portion 3 side, and an introduction-side leg portion 72 disposed on the introduction portion 2 side of the closed space S. The frame-shaped leg portion 7 functions as a sealing portion which provides sealing between the liquid feeding device 1 and the chip 4 when the liquid feeding device 1 is placed on the chip 4.

The pair of side leg portions 71, 71 forms side walls which define end portions of the closed space S in the width direction, and the introduction-side leg portion 72 forms a side wall which defines an end portion of the chamber 22 on one end portion 1a side. The discharge port 31 is provided on the discharge portion 3 side of the pair of side leg portions 71, 71 at least between the pair of side leg portions 71, 71 so as to extend along the width direction D of the closed space S as viewed in a plan view.

The frame-shaped leg portion 7 has no leg portion on the discharge portion 3 side (FIG. 1A, FIG. 1B). However, it may be configured such that the extension length of the discharge port 31 is reduced, and the frame-shaped leg portion 7 has a discharge-side leg portion disposed on the discharge portion 3 side. In this case, the above-mentioned discharge-side leg portion is provided in the form of a bridge shape, for example, and a space formed below the discharge-side leg portion forms the discharge port 31. The shape of the discharge port 31 as viewed in a side view may be a polygonal shape, such as a rectangular shape, or may be an arch shape.

Intermittent portions may be formed on the discharge portion 3 side of the frame-shaped leg portion 7 such that the intermittent portions are formed intermittently along the extending direction of the frame-shaped leg portion 7. In this case, the intermittent portions formed on the frame-shaped leg portion 7 form the discharge port 31, and the discharge port 31 is provided between the pair of side leg portions 71, 71.

With such a configuration, when a pressing force is applied to the liquid feeding device 1 from the upper portion 1c side toward the lower portion 1d side, the discharge portion 3 side of the frame-shaped leg portion 7 can be elastically deformed more easily than the introduction portion 2 side of the frame-shaped leg portion 7.

FIG. 2 is a view for describing a closed flow path formed in the closed space S on the chip 4 by the liquid feeding device 1 shown in FIG. 1, wherein FIG. 2A is a cross-sectional view, and FIG. 2B is a plan view.

As shown in FIG. 2A, when the liquid feeding device 1 is placed on the upper surface 4a of the chip 4 on which the plurality of wells 4b are formed, the closed space S is formed between the upper surface 4a of the chip 4 and the inclined surface 5a of the liquid feeding device 1, and a closed flow path 8 of a liquid which flows from the introduction portion 2 side toward the discharge portion 3 side is formed in the closed space S. When a liquid is supplied to the closed flow path 8, a liquid flow F with substantially the same velocity in the width direction of the closed space S is generated from the introduction portion 2 side toward the discharge portion 3 side (FIG. 2B).

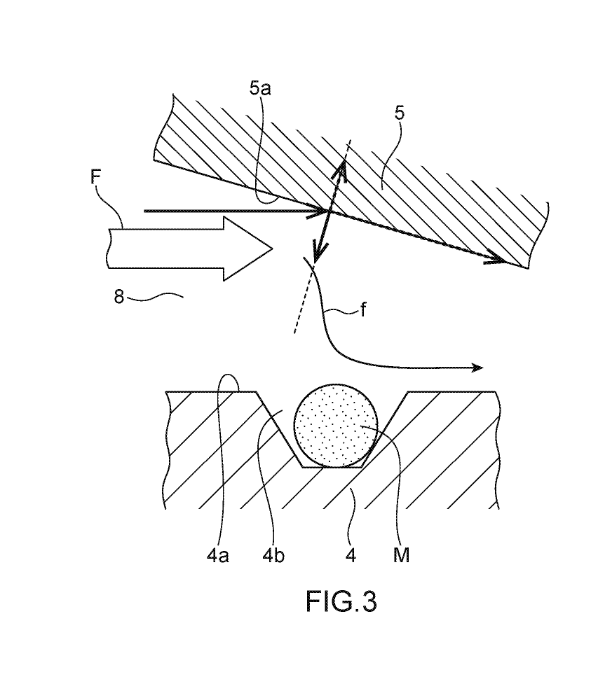

At this point of operation, when an area in the vicinity of the well 4b is microscopically observed, as shown in FIG. 3, the liquid flow F impinges on the inclined surface 5a in the closed flow path 8 so that a reaction force is generated in the direction perpendicular to the inclined surface 5a due to impingement. By the action of this reaction force, a liquid flow f which flows from the inclined surface 5a toward the upper surface 4a of the chip 4, particularly, from the inclined surface 5a toward the wells 4b on the chip 4 is formed. The liquid flow f applies a force by which cells M are pushed into the wells 4b to the cells M from above.

Accordingly, for example, when a liquid is supplied to the closed flow path 8 in a state where a cell M is stored in each well of the chip 4, the closed flow path 8 generates a liquid flow f which flows from the inclined surface 5a toward the wells 4b on the chip 4 due to impingement between the liquid flow F and the inclined surface 5a. Accordingly, the cells M are pushed into the wells 4b by the liquid flow f, thus remaining in the wells 4b and hence, escape of the cells M from the wells 4b can be inhibited.

When a liquid is supplied to the closed flow path 8 in a state where a cell M is not stored in each well of the chip 4, in the same manner as the above-mentioned case, the closed flow path 8 generates a liquid flow f which flows from the inclined surface 5a toward the wells 4b on the chip 4 due to impingement between the liquid flow F and the inclined surface 5a. Accordingly, the liquid is pushed into the wells 4b by the liquid flow f so that air bubbles and the like in the wells 4b can be expelled from the wells 4b and hence, the liquid is sufficiently supplied into the wells 4b.

The inclined surface 5a is provided such that a gap formed between the inclined surface 5a and the chip 4 is reduced from the introduction portion 2 side toward the discharge portion 3 side and hence, the flow velocity of the liquid flow F increases from the introduction portion 2 side toward the discharge portion 3 side. Accordingly, by the liquid flow f generated due to impingement between the liquid flow F and the inclined surface 5a, the cells M in the wells 4b positioned on the downstream side can be more strongly pushed into the wells 4b and hence, escape of the cells M from the wells 4b can be inhibited.

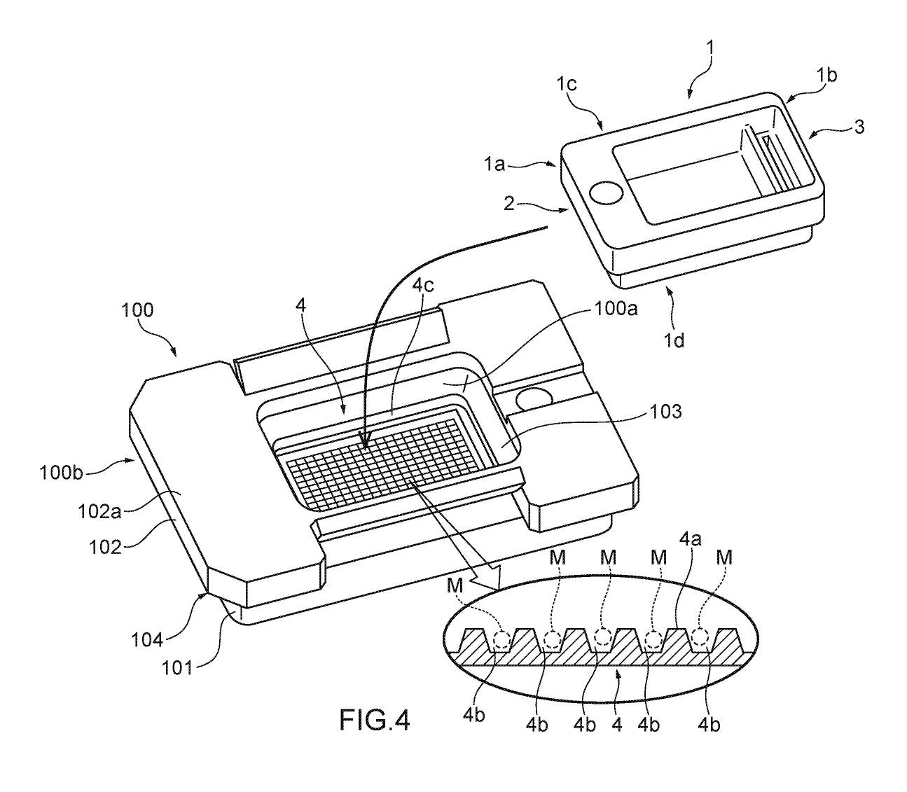

FIG. 4 is a perspective view showing a holder which fixes the liquid feeding device 1 shown in FIG. 1 on the chip 4.

As shown in the drawing, a holder 100 includes fixing members 101, 102 having a frame-like body structure which can be divided into two parts in the vertical direction. The holder 100 has an opening portion 103 at a center portion as viewed in a plan view. An outer peripheral portion 4c of the chip 4 is sandwiched by inner peripheral portions 100a of the holder 100 in the vertical direction. With such a configuration, the chip 4 is fixed such that the wells 4b are exposed to the opening portion 103 of the holder 100.

Inserting the liquid feeding device 1 into the opening portion 103 so as to place the liquid feeding device 1 on the chip 4, which is fixed to the holder 100, allows the liquid feeding device 1 to be supported on the chip 4. Further, the movement of the liquid feeding device 1 in the lateral direction is restricted by the holder 100 so that the liquid feeding device 1 is positioned in the in-plane direction of the chip 4.

To provide sealing between the liquid feeding device 1 and the chip 4, the liquid feeding device 1 is placed on the chip 4 in a state where the height of the upper portion 1c of the liquid feeding device 1 and the height of an upper surface 102a of the fixing member 102 are controlled and adjusted. With such a configuration, the upper portion 1c of the liquid feeding device 1 is brought into pressure contact with the upper surface 102a of the fixing member 102 by a fixing device described later with a desired pressing allowance.

The holder 100 also has a stepped portion 104 on an outer peripheral portion 100b, and this stepped portion 104 is engaged with the fixing device described later. The length and width of the fixing member 102 are larger than the length and width of the fixing member 101. The stepped portion 104 is formed on the outer peripheral portion 100b of the holder 100 in a state where the fixing members 101, 102 are assembled in the vertical direction.

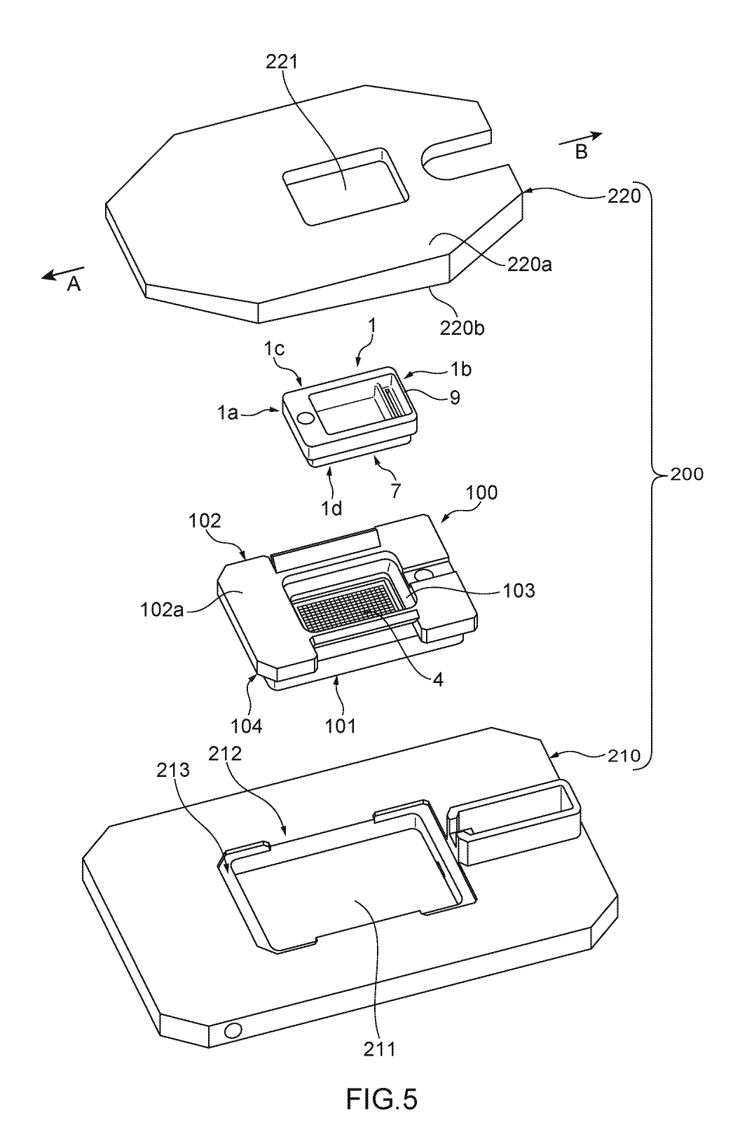

FIG. 5 is an exploded perspective view showing the fixing device which fixes the holder 100 shown in FIG. 4 at the time of feeding a liquid onto the chip 4 using the liquid feeding device 1.

As shown in the drawing, a fixing device 200 includes a base member 210 and a cover member 220 which can be divided into two parts in the vertical direction. The base member 210 is formed of a plate-like body having a substantially polygonal shape as viewed in a plan view, and the base member 210 has an opening portion 211 at a center portion thereof. In the same manner, the cover member 220 is also formed of a plate-like body having a polygonal shape as viewed in a plan view, and the cover member 220 has an opening portion 221 at a center portion thereof. The thickness of the cover member 220 is gradually increased from one end portion 1a side toward the other end portion 1b side of the liquid feeding device 1. When the cover member 220 is disposed with an upper surface 220a extending horizontally, a lower surface 220b of the cover member 220 is disposed in a downwardly inclined manner.

The base member 210 has a stepped portion 213 on an inner peripheral portion 212 thereof. The stepped portion 213 is engaged with the stepped portion 104 of the holder 100 in a state where the holder 100 is inserted into the opening portion 211. With such a configuration, the movement of the holder 100 in the lateral direction is restricted. Further, the cover member 220 is placed on the base member 210 in a state where the stepped portion 213 and the stepped portion 104 are engaged with each other. Accordingly, the movement of the holder 100 in the vertical direction is restricted and, at the same time, the movement of the liquid feeding device 1 in the vertical direction is also restricted.

The liquid feeding device 1 may also have a pressing surface 9. The pressing surface 9 is formed on a surface of the liquid feeding device which is not in contact with the chip 4, for example, on the upper portion 1c of the liquid feeding device 1 which is disposed on a side opposite to the frame-shaped leg portion 7, and the pressing surface 9 is capable of being pressed toward the upper surface 4a of the chip 4. The pressing surface 9 is a frame-shaped surface formed corresponding to the entire frame-shaped leg portion 7, for example. However, the pressing surface 9 may also be formed on the discharge portion 3 side. Further, the lower surface 220b of the cover member 220 may also have a projecting surface formed at the position which corresponds to the pressing surface 9, and coming into contact with the pressing surface 9. When the cover member 220 is placed on the base member 210, the pressing surface 9 is pressed by the lower surface 220b of the cover member 220 with a desired pressing amount so that the liquid feeding device 1 is brought into pressure contact with the chip 4. At this point of operation, the discharge port 31 is formed on the discharge portion 3 side of the pair of side leg portions 71, 71 so that there is no leg portion which comes into contact with the upper surface 4a of the chip 4 on the discharge portion 3 side. Accordingly, the discharge side of the frame-shaped leg portion 7 is significantly deflected relative to the introduction side of the frame-shaped leg portion 7 and hence, the degree of inclination of the inclined surface 5a of the upper wall 5 with respect to the upper surface 4a of the chip 4 can be adjusted. To adjust the degree of inclination of the inclined surface 5a, the pressing amounts may be set relative to each other such that the pressing amount on the discharge portion 3 side is larger than the pressing amount on the introduction portion 2 side. Alternatively, it may be also configured such that the pressing amount on the introduction portion 2 side is set to zero or approximately zero, and only the setting of the pressing amount on the discharge portion 3 side is performed.

[Method for Feeding liquid Using Liquid Feeding Device]

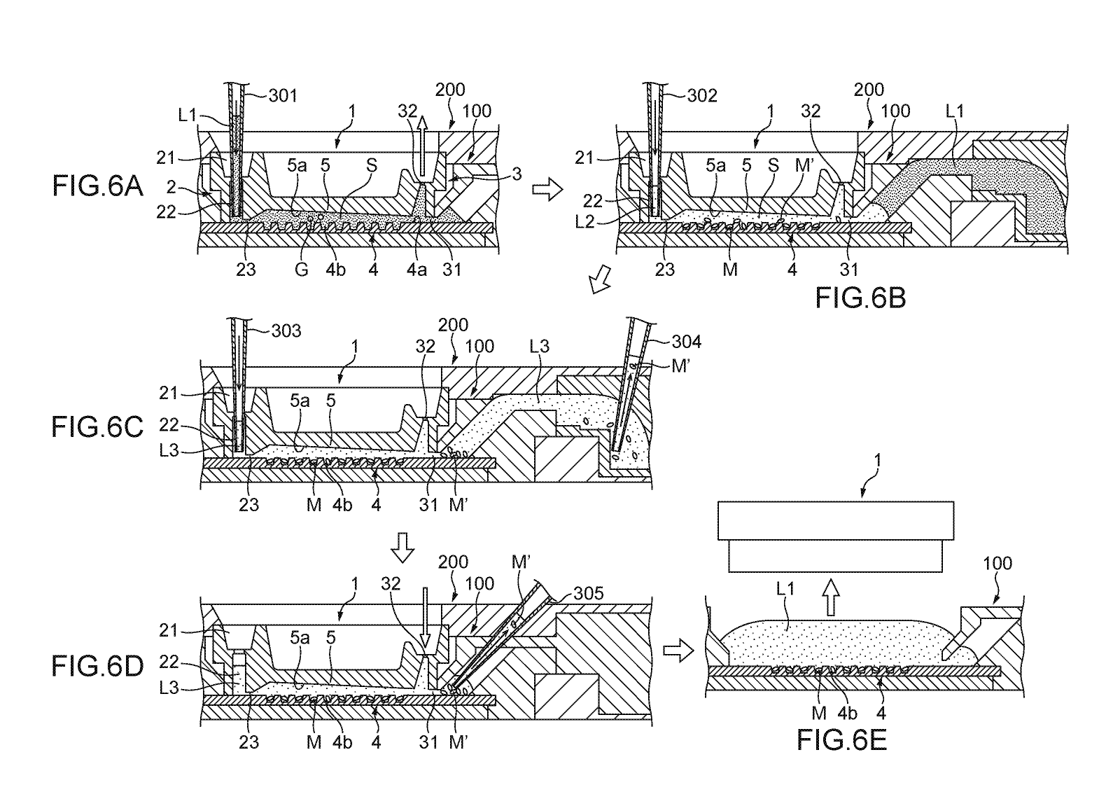

FIG. 6A to FIG. 6E are cross-sectional views for describing a method for feeding a liquid using the liquid feeding device shown in FIG. 1. First, the chip 4 is fixed to the holder 100 and, at the same time, the holder 100 and the liquid feeding device 1 are further fixed to the fixing device 200 in a state where the liquid feeding device 1 is placed on the chip 4. With such operations, the closed space S is formed between the inclined surface 5a of the liquid feeding device and the upper surface 4a of the chip 4, on which the plurality of wells 4b are formed, such that a gap formed between the inclined surface 5a and the chip 4 is reduced from the side where the liquid is introduced (introduction portion 2 side) toward the side where the liquid is discharged (discharge portion 3 side). At this point of operation, the frame-shaped leg portion 7 of the liquid feeding device 1 abuts on or is brought into pressure contact with the chip 4.

Next, a buffer L1 is supplied to the chamber 22 with a tubular member 301 inserted through the supply port so that the buffer L1 is introduced into the closed space S through the first slit portion 23. With such an operation, the chamber 22, the first slit portion 23, and the closed space S are filled with the buffer L1. Further, by introducing the buffer L1, air or air bubbles in the chamber 22, the first slit portion 23 and the closed space S are removed to the outside through the discharge port 31 or the second slit portion 32.

Next, a cell suspension L2 containing a plurality of cells M is supplied to the chamber 22 with a tubular member 302 inserted through the supply port 21 so that the cell suspension L2 is introduced into the closed space S through the first slit portion 23 (FIG. 6B). With such an operation, the plurality of cells M contained in the cell suspension L2 are stored in the plurality of wells 4b on a one-to-one basis, and the remaining cells M′ which are not stored in the wells 4b (also referred to as cells outside the wells) are deposited on the upper surface 4a of the chip 4 or the like.

Next, a cleaning solution L3 is supplied to the chamber 22 with a tubular member 303 inserted through the supply port 21 so that the cleaning solution L3 is introduced into the closed space S through the first slit portion 23, thus forming a closed flow path which flows from the above-mentioned introduction side toward the discharge side between the upper surface 4a of the chip 4 and an inclined surface in the closed space S (FIG. 6C). At this point of operation, due to impingement between a liquid flow F of the cleaning solution L3 and the inclined surface 5a of the upper wall 5, a liquid flow f which flows from the inclined surface 5a toward the cells M is generated on the upper surface 4a of the chip 4 (see FIG. 3). With the formation of such a flow, the cells M stored in the wells 4b are hold in the wells 4b without being rinsed away by the cleaning solution L3. On the other hand, the cells M′ on the upper surface 4a of the chip 4 are rinsed away by the cleaning solution L3. The second slit portion 32 is formed with a larger flow path resistance compared to the discharge port 31. Accordingly, the cleaning solution L3 containing some cells M′ is discharged to the outside through the discharge port 31 without causing a backflow to the outside through the second slit portion 32, and the cleaning solution L3 is removed through a tubular member 304 as a discharged solution. The cleaning solution L3 may be a buffer or a cell culture solution, for example.

Next, a tubular member 305 is inserted into an area in the vicinity of the discharge port 31 so as to remove the cells M′ accumulating at the discharge port 31 (FIG. 6D). During suction performed by the tubular member 304, outside air is introduced into the closed space S through the second slit portion 32. With such a configuration, the cleaning solution L3 in the closed space S is not sucked so that it is possible to maintain a state where the cells M are stored in the wells 4b.

Thereafter, the fixing device 200 is detached, and the liquid feeding device 1 is removed from the chip 4 and, then, the upper surface 4a and the inside of the wells 4b are filled with the buffer L1 (FIG. 6E). With such operations, preparation of the chip 4 storing the buffer L1 such that one cell M is included in each well is completed.

In the above-mentioned method for feeding a liquid, supply of a variety of liquids to the supply port 21 and discharge of the variety of liquids from the discharge port 31 may be manually performed using a pipette or the like. Alternatively, it may be configured such that an introduction pipe and a discharge pipe provided to facility equipment, such as a screening device described later, are connected to the supply port 21 and the discharge port 31 respectively, and a variety of liquids is automatically supplied to the supply port 21, and is automatically discharged from the discharge port 31.

[Configuration of Screening Device]

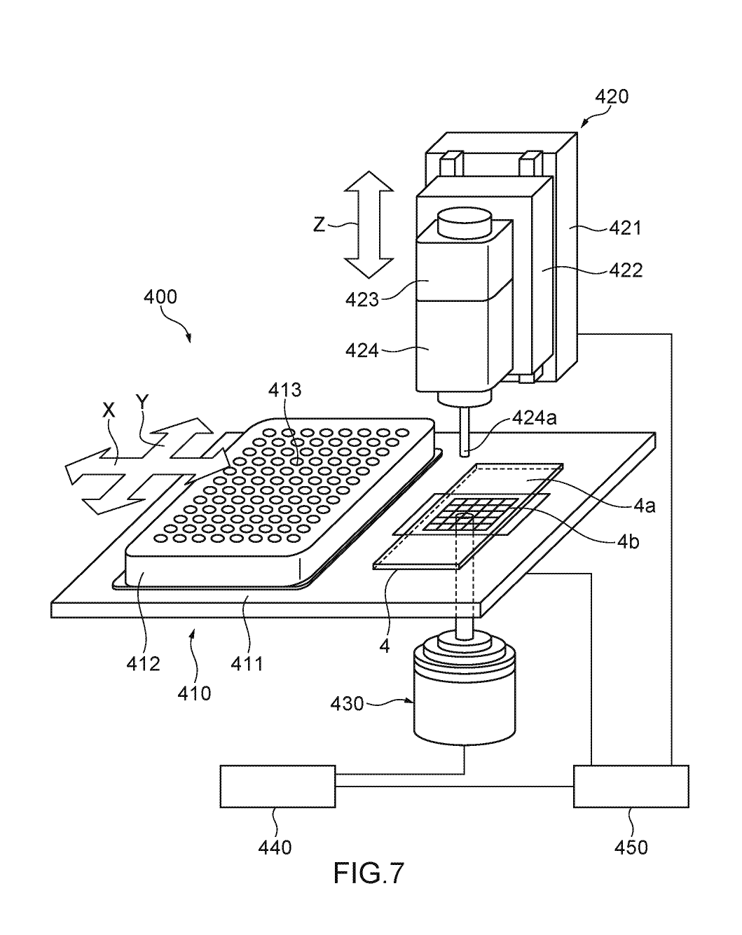

After the method for feeding a liquid shown in FIG. 6 is performed, target specimens are identified and sorted from fine particles on the chip 4 by the screening device shown in FIG. 7, for example. The screening device is a device which searches for predetermined fine particles which form target specimens based on fluorescence emitted from a plurality of fine particles, such as cells, in the chip 4 so as to selectively suck and collect fine particles which satisfy collection conditions from the chip 4.

A screening device 400 includes a moving unit 410, a collection unit 420, a measurement unit 430, an image analysis unit 440 (analysis unit), and a control unit 450, for example.

The moving unit 410 includes a mounting table 411 which is movable along the X direction and/or the Y direction so that the moving unit 410 can perform positioning of a storage plate 412 and the chip 4 disposed on the mounting table 411 by moving along the X direction and/or the Y direction. The storage plate 412 is a plate-like member, and a large number of wells 413 are arranged on the storage plate 412 equidistantly in a matrix pattern along the X direction and the Y direction. These wells 413 are collection accommodating portions which can separately collect and accommodate fine particles which are sequentially discharged when fine particles, such as biological cells, are discharged sequentially from a suction/delivery capillary described later.

The collection unit 420 includes a base unit 421 fixed to a device body, and an operation unit 422 mounted on the base unit 421 in a movable manner in the Z direction. The operation unit 422 includes an actuator unit 423 (pump), and a suction/delivery capillary 424 which sorts an identified fine particle as a target specimen. The suction/delivery capillary 424 can suck one fine particle from a selected well out of the plurality of wells 4b, that is, from a well storing a fine particle which satisfies predetermined collection conditions. The suction/delivery capillary 424 can also deliver the above-mentioned selected one fine particle to a predetermined well 413 of the storage plate 412.

The measurement unit 430 irradiates the chip 4 and the fine particles stored in the chip 4 with light emitted from at least one light source. With such an operation, the measurement unit 430 acquires, from transmitted light, reflected light or fluorescence, shape and position information, and brightness information, such as fluorescence/chemiluminescence, at a resolution finer than an average size of an individual fine particle. At the same time, the measurement unit 430 acquires information, such as the shape of the chip 4 per se and position coordinates and the size of the wells 4b on the chip 4.

The image analysis unit 440 analyzes measured shape information and optical information so as to acquire at least data for checking the presence of a fine particle which satisfies brightness conditions, which can be set by a measurer, in each well 4b. The image analysis unit 440 collates position coordinate information of the well 4b and optical information of fluorescence/chemiluminescence from the transmitted light or reflected light, thus extracting optical information of the fine particles. Further, the measurement unit 430 has an automatic focus function so that the measurement unit 430 performs a measurement at a predetermined position in a focused state and, at the same time, can determine a positional relationship between a distal end portion 424a of the suction/delivery capillary 424 and the upper surface 4a of the chip 4 by performing automatic focus with respect to the distal end portion 424a and the upper surface 4a.

The control unit 450 centrally controls a variety of constitutional elements of the screening device 400. The control unit 450 also detects positions of the wells 4b each storing a fine particle which satisfies collection conditions and emits fluorescence of maximum brightness. By applying a control drive signal to the moving unit 410, the control unit 450 can cause the well 4b of the chip 4 or the well 413 of the storage plate 412 to be positioned just below the distal end portion 424a of the suction/delivery capillary 424.

In the screening device 400 having such a configuration, as many fine particles as possible are stored in the wells 4b of the chip 4 on a one-to-one basis in performing a screening process and hence, collection efficiency of fine particles which form target specimens can be improved. Accordingly, storing a large number of fine particles in the plurality of wells 4b on a one-to-one basis using the liquid feeding device 1 by the above-mentioned method for feeding a liquid can improve collection efficiency of target specimens in the screening process. In this case, the screening device 400 may include the liquid feeding device 1, which is detachably mounted on the screening device 400, as a constitutional element. Alternatively, one system formed of the screening device 400 and the liquid feeding device may be provided.

As described above, according to the present embodiment, the liquid feeding device 1 has the upper wall 5 provided so as to oppose the upper surface 4a of the chip 4, and configured to define the closed space S. The upper wall 5 has the inclined surface 5a which is provided such that a gap formed between the inclined surface 5a and the chip 4 is reduced from the introduction portion 2 side toward the discharge portion 3 side and hence, it is possible to form the closed flow path 8 of a liquid which flows from the introduction portion 2 side toward the discharge portion 3 side in the closed space S. Accordingly, when a liquid is supplied into the closed space S, a liquid flow f which flows from the inclined surface 5a toward the upper surface 4a of the chip 4, particularly, from the inclined surface 5a toward the wells 4b is formed and hence, the cells M in the wells 4b receive a pressure from above due to the liquid flow f, thus preventing the cells M from escaping from the wells 4b. Further, by increasing the flow velocity of the liquid flow F in the closed space S, an accurate reaction between a reagent and the cells M can be measured, and excess cells M′ which are not stored in the wells 4b can be effectively cleaned, thus reducing the number of times of cleaning. Therefore, collection efficiency of the cells M which form target specimens on an individual particle basis can be improved while improvement of workability is realized. Particularly, at the time of cleaning the chip 4, the cells M stored in the respective wells are prevented from easily escaping from the wells 4b and hence, when rare cells are collected as target specimens, the number of cells M stored on the chip 4 is increased so that the rare cells can be efficiently collected.

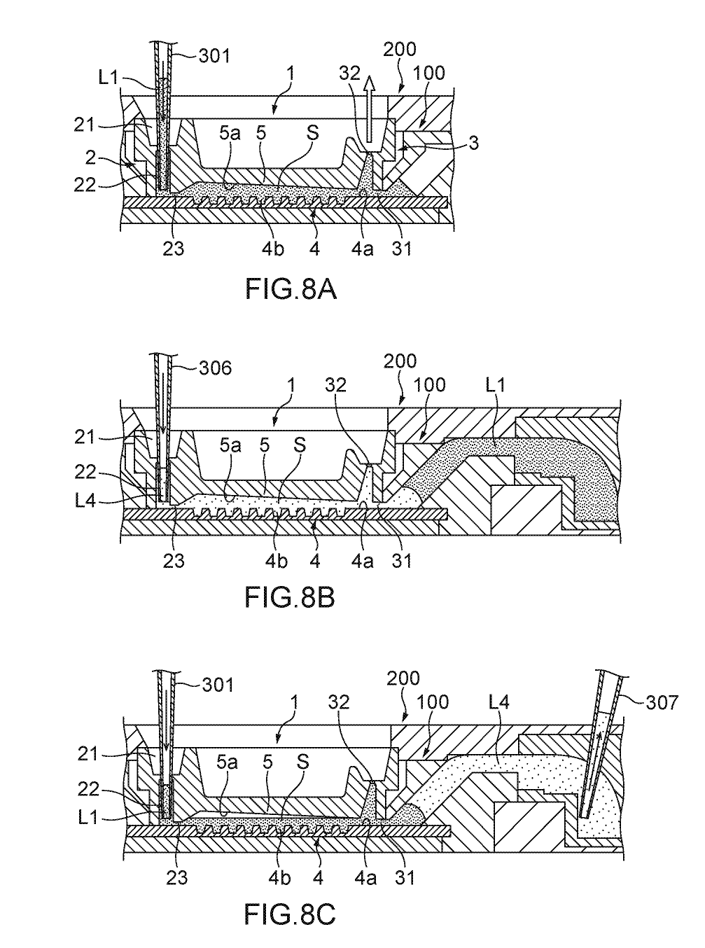

FIG. 8A to FIG. 8C are cross-sectional views for describing a variant of the method for feeding a liquid using the liquid feeding device shown in FIG. 1. In the above-mentioned embodiment, the description has been made with respect to the liquid feeding method by focusing on a cleaning process which is performed after cells M are stored in wells. However, the application of the present disclosure is not limited to such a case. As shown in FIG. 8, the method for feeding a liquid of the present disclosure may also be applied in performing a surface treatment on the chip 4 with the wells 4b filled with a reagent. The configuration shown in FIG. 8 is basically equal to the configuration shown in FIG. 6 and hence, the repeated description is omitted.

To be more specific, in the same manner as the method for feeding a liquid shown in FIG. 6, first, a liquid feeding device 1 is placed on a chip 4, and a closed space S is formed between an inclined surface 5a of the liquid feeding device and an upper surface 4a of the chip 4, on which a plurality of wells 4b are formed, such that a gap formed between the inclined surface 5a and the chip 4 is reduced from the side where the liquid is introduced toward the side where the liquid is discharged. Then, a buffer L1 is supplied to a chamber 22 with a tubular member 301 inserted through a supply port 21 so that the buffer L1 is introduced into the closed space S through a first slit (FIG. 8A). With such operations, the chamber 22, the first slit portion 23, and the closed space S are filled with the buffer L1. Further, by introducing the buffer L1, air or air bubbles in the chamber 22, the first slit portion 23 and the closed space S are removed to the outside through a discharge port 31 or a second slit portion 32.

Next, a reagent L4 is supplied to the chamber 22 with a tubular member 306 inserted through the supply port 21 so that the reagent L4 is introduced into the closed space S through the first slit 23, thus forming a closed flow path which flows from the above-mentioned introduction side toward the discharge side between the upper surface 4a of the chip 4 and the inclined surface 5a in the closed space S (FIG. 8B). The reagent L4 may be a liquid containing a primary antibody to be fixed to a surface of wells of an immunochamber, or a liquid for modifying the surface of the wells so as to cause the surface of the wells to have hydrophilicity with respect to a cell suspension or the like, for example. At this point of operation, due to impingement between a liquid flow F of the reagent L4 and the inclined surface 5a of the upper wall 5, a liquid flow f which flows from the inclined surface 5a toward the wells 4b is generated in the closed space S (see FIG. 2). The liquid flow f causes the reagent L4 to arrive at inner wall surfaces of the wells 4b so that a primary antibody can be sufficiently fixed to the inner wall surfaces of the wells 4b and hence, modification treatment can be sufficiently performed on the inner wall surfaces of the wells 4b.

Next, a buffer L1 is supplied to the chamber 22 with the tubular member 301 inserted through the supply port 21 so that the buffer L1 is introduced into the closed space S through the first slit 23 so as to clean the inside of the closed space S, thus removing the reagent L4 from the inside of the closed space S (FIG. 8C). The reagent L4 is discharged to the outside through the discharge port 31, and is removed by a tubular member 307 as discharged solution.

As described above, according to this variant, when the reagent L4 is introduced into the closed space S, a liquid flow f which flows from the inclined surface 5a toward the wells 4b of the chip 4 is generated. The liquid flow f causes the reagent L4 to arrive at the entire inner wall surfaces of the wells 4b so that surface treatment can be sufficiently performed on the entire inner wall surfaces of the wells 4b. The liquid flow f also causes the reagent L4 to arrive at the entire wells 4b arranged on the chip 4 in an aligned manner in a matrix pattern. Accordingly, wastage of cells M which form target specimens is inhibited and hence, collection efficiency of the cells M on an individual particle basis can be further improved.

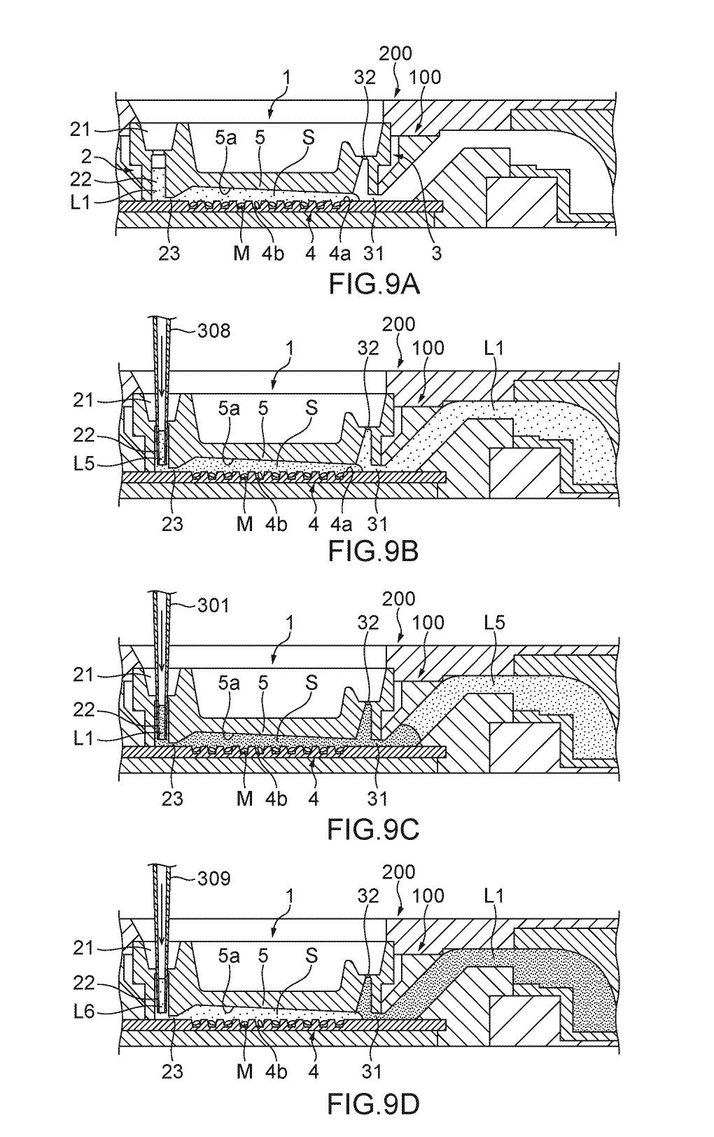

FIG. 9A to FIG. 9D are cross-sectional views for describing another variant of the method for feeding a liquid using the liquid feeding device shown in FIG. 1. In the above-mentioned variant, the description has been made with respect to the method for performing, using the method for feeding a liquid of the present disclosure, surface treatment with the wells 4b filled with the reagent. However, the application of the present disclosure is not limited to such a case. The method for feeding a liquid of the present disclosure may also be applied in supplying a reagent to cells M in wells 4b so as to cause the cells M to react. The configuration shown in FIG. 9 is basically equal to the configuration shown in FIG. 8 and hence, the repeated description is omitted.

In this another variant, first, steps substantially equal to the steps shown in FIG. 6A to FIG. 6E are performed, thus bringing about a state where cells M are stored in a plurality of wells 4b on a one-to-one basis (FIG. 9A). Thereafter, a reagent L5 is supplied to a chamber 22 with a tubular member 306 inserted through a supply port 21 so that the reagent L5 is introduced into a closed space S through the first slit 23, thus forming a closed flow path which flows from the above-mentioned introduction side toward the discharge side between an upper surface 4a of a chip 4 and an inclined surface 5a in the closed space S (FIG. 9B). The reagent L5 may be a liquid containing a fluorescent molecule, such as a secondary antibody with fluorescence, which bonds to a substance to be produced which is produced from cells, or a stimulant A which reacts to a specific cell, for example. The stimulant A may be a sweet extract or the like which stimulates a sense of taste in a taste cell, for example. At this point of operation, due to impingement between a liquid flow F of the reagent L5 and the inclined surface 5a of an upper wall 5, a liquid flow f which flows from the inclined surface 5a toward the wells 4b is generated in the closed space S (see FIG. 2). The liquid flow f causes the reagent L5 to arrive at cells M stored in the wells 4b with certainty, thus causing the cells M in the wells 4b to accurately react. For example, in the case where the reagent L5 is the stimulant A which reacts to a cell, by measuring and analyzing optical information while feeding the stimulant A, the same stimulus can be applied to the cells M in the wells 4b. Accordingly, the degree of reaction can be accurately acquired so that target cells can be specified.

Next, a buffer L1 is supplied to the chamber 22 with a tubular member 301 inserted through the supply port so that the buffer L1 is introduced into the closed space S through the first slit 23 so as to clean the inside of the closed space S, thus removing the reagent L5 from the inside of the closed space S (FIG. 9C). The reagent L5 is discharged to the outside through a discharge port 31.

Next, a reagent L6 is supplied to the chamber 22 with a tubular member 309 inserted through the supply port 21 so that the reagent L6 is introduced into the closed space S through the first slit 23, thus filling the closed space S with the reagent L6 (FIG. 9D). The reagent L6 may be a stimulant B which is different from the stimulant A, and reacts to a specific cell, for example. The stimulant B may be a sour extract or the like which stimulates a sense of taste in a taste cell, for example. As described above, with the use of the specific stimulant A and stimulant B, target cells can be selected in a more complex manner.

As described above, according to this variant, when the reagent L5 is introduced into the closed space S, the liquid flow f which flows from the inclined surface 5a toward the wells 4b of the chip 4 is generated. The liquid flow f causes the reagent L5 to arrive at the cells M stored in the wells 4b, thus causing the cells M in the wells 4b to accurately react. The liquid flow f also causes the reagent L5 to arrive at all cells M stored in the plurality of wells 4b on the chip 4 on a one-to-one basis. Accordingly, wastage of cells M which form target specimens is inhibited and hence, collection efficiency of the cells M on an individual particle basis can be further improved.

The liquid feeding device and the method for feeding a liquid according to the present embodiment have been described heretofore. However, the present disclosure is not limited to the described embodiment, and various variations and modifications are conceivable based on the technical concept of the present disclosure.

In the above-mentioned embodiment, the upper wall 5 of the liquid feeding device 1 has the inclined surface 5a. However, the present disclosure is not limited to such a configuration. It may be configured such that the upper wall has no inclined surface, and the upper wall forms an inclined surface when the liquid feeding device receives an external force.

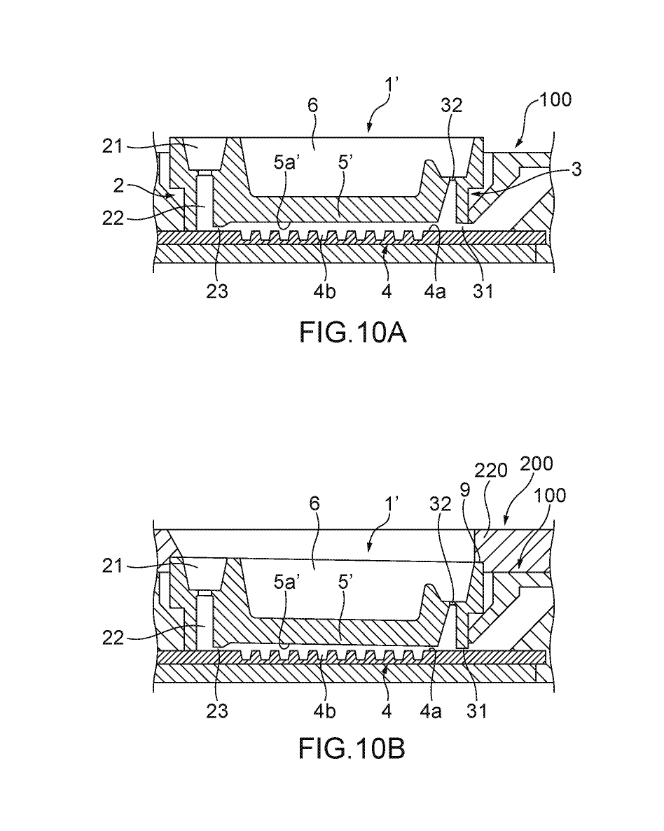

For example, as shown in FIG. 10A, it may be configured such that a liquid feeding device 1′ is provided so as to oppose an upper surface 4a of a chip 4, and has an upper wall 5′ which defines a closed space S, and the upper wall 5′ has a surface 5a′ which is substantially parallel to the chip 4. When the liquid feeding device 1′ is in a state of being placed on the chip 4 fixed to a holder 100, the surface 5a′ is substantially parallel to the upper surface 4a of the chip 4. However, when the holder 100 and the liquid feeding device 1′ are fixed to a fixing device 200, a pressing surface 9 of the liquid feeding device 1′ is pressed downward by a cover member 220 so that the discharge side of a frame-shaped leg portion 7 deflects the most. Accordingly, the surface 5a′ is disposed in an oblique manner such that a gap formed between the surface 5a′ and the chip 4 is reduced from the introduction portion side toward the discharge portion 3 side (FIG. 10B). Also with this configuration, a closed flow path of a liquid which flows from the introduction portion 2 side toward the discharge portion 3 side is formed in the closed space S so that collection efficiency of cells M which form target specimens on an individual particle basis can be improved while improvement of workability is realized.

1. A liquid feeding device capable of forming a closed space between the liquid feeding device and an upper surface of a chip on which at least one well is formed for storing a fine particle, the liquid feeding device comprising:
an introduction portion disposed on one end side of the liquid feeding device, and configured to introduce a liquid into the closed space;
a discharge portion disposed on another end side of the liquid feeding device, and configured to discharge the liquid supplied into the closed space; and
an upper wall provided so as to oppose the upper surface of the chip, and configured to define the closed space,
wherein the upper wall has an inclined surface which is provided such that a gap formed between the inclined surface and the chip is reduced from an introduction portion side toward a discharge portion side.

2. The liquid feeding device according to claim 1, wherein
a closed flow path of a liquid which flows from the introduction portion side toward the discharge portion side is formed in the closed space.

3. The liquid feeding device according to claim 2, wherein
the closed flow path generates a liquid flow which flows from the inclined surface toward the upper surface of the chip.

4. The liquid feeding device according to claim 1, wherein the inclined surface is a tilted surface having a gradient with respect to the upper surface of the chip, and
a cross-sectional area of the closed space in a direction perpendicular to a tilt direction of the tilted surface is gradually reduced from the introduction portion side toward the discharge portion side.

5. The liquid feeding device according to claim 1, wherein the chip has a plurality of wells arranged in an aligned manner on the upper surface of the chip, and
the inclined surface is formed at a position which corresponds to all of the plurality of wells as viewed in a plan view.

6. The liquid feeding device according to claim 1, wherein
the introduction portion has:
a supply port into which a liquid is supplied from an outside;
a chamber which is coupled to the supply port, and which extends in a width direction of the closed space as viewed in a plan view; and
a first slit portion which makes the chamber and the closed space communicate with each other, and which extends along the width direction of the closed space as viewed in a plan view.

7. The liquid feeding device according to claim 6, wherein a height of the first slit portion is set smaller than a maximum height of the closed space.

8. The liquid feeding device according to claim 1, wherein
the discharge portion has:
a discharge port which is coupled to the closed space, and through which a liquid in the closed space is discharged to an outside; and
a second slit portion which is formed between the closed space and the discharge port, and which extends upward from the closed space.

9. The liquid feeding device according to claim 8, further comprising a frame-shaped leg portion which abuts on the upper surface of the chip, and which is provided so as to surround the closed space, wherein
the frame-shaped leg portion includes: a pair of side leg portions disposed on both sides of the closed space along a direction from the introduction portion side toward the discharge portion side; and an introduction-side leg portion disposed on the introduction portion side, and
an intermittent portion is formed in the discharge portion side of the frame-shaped leg portion.

10. The liquid feeding device according to claim 9, wherein
the intermittent portion formed in the frame-shaped leg portion forms the discharge port, and
the discharge port is provided between the pair of side leg portions.

11. The liquid feeding device according to claim 10, further comprising a pressing surface on a surface of the liquid feeding device which is not in contact with the chip, the pressing surface being capable of being pressed toward the upper surface of the chip, wherein the pressing surface is disposed on the discharge portion side.

12. The liquid feeding device according to claim 1, wherein the liquid feeding device is integrally molded by an elastic material.

13. A method for feeding a liquid where a closed space which allows introduction and discharge of a liquid is formed on an upper surface of a chip on which at least one well is formed for storing a fine particle, thus allowing introduction and discharge of the liquid onto and from the upper surface of the chip, the method for feeding a liquid including
a closed space is formed such that the closed space is reduced from a side where the liquid is introduced toward a side where the liquid is discharged, and
the liquid is introduced into the closed space, thus forming a closed flow path which flows from the introduction side toward the discharge side in the closed space.

14. The method for feeding a liquid according to claim 13, wherein a liquid flow which flows toward the upper surface of the chip is generated by the closed flow path.
